(19) United States
(12) Patent Application Publication
(10) Pub. No.: US 2022/0251490 A1
(43) Pub. Date: Aug. 11, 2022

(54) METHOD FOR PRODUCING AND/OR ARRANGING CELL CULTURES

(71) Applicant: AXENOLL LIFE SCIENCES AG, Zurich (CH)

(72) Inventors: David L. Deck, Zurich (CH); Srdan Vasic, Zurich (CH)

(21) Appl. No.: 17/628,097

(22) PCT Filed: Jul. 16, 2020
(86) PCT No.: PCT/EP2020/070221

§ 371 Date: Jan. 18, 2022

(30) Foreign Application Priority Data
Jul. 18, 2019 (EP) 19187054.2

(51) Int. Cl. C12M 1/12; C12M 3/00

(52) U.S. Cl. CPC C12M 25/14; C12M 25/06; C12M 21/08

(57) ABSTRACT

Method for producing and/or arranging cell cultures and/or cell agglomerations, in particular for carrying out medical and/or pharmacological examinations, in which a printing medium containing living cells is provided and in which the printing medium is printed through a printing screen and/or a printing stencil.

### CROSS REFERENCE TO RELATED APPLICATIONS

This application is a U.S. National Stage of International Patent Application No. PCT/EP2020/070221 filed Jul. 16, 2020, which claims the benefit of priority of European Patent Application No. EP19187054.2 filed Jul. 18, 2019, the respective disclosures of which are each incorporated herein by reference in their entireties.

### FIELD OF THE DISCLOSURE

The invention relates to a method for producing and/or arranging cell cultures and/or cell agglomerations. The invention also relates to an arrangement of a plurality of cell cultures and/or cell agglomerations.

### BACKGROUND

For the development of cell cultures, it is known to fill or apply living cells in so-called cell carriers. Such cell carriers can be designed, for example, as “arrays” or “hanging drop plates”. Cell cultures in cell carriers can be used, for example, for medical or pharmacological examinations.

The filling or application of such cell carriers can be done, for example, using pipettes or pipette systems. In this case, however, the cells may be exposed to high shear stress, which may reduce the survival rate of the cells. Depending on the design of the pipettes or pipette systems, filling the cell carriers with cells or applying the respective cells to the cell carriers may require many individual steps, thus extending the overall process time of filling or application. The remaining examination time of the respective cell culture can thus be reduced, since several cell divisions may already have taken place by the time filling or application is completed.

### SUMMARY

Against the above mentioned background the task of the present invention was to provide a method for producing and/or arranging cell cultures and/or cell agglomerations that can be performed with improved productivity while reducing the risk of cell damage. Likewise, the task was to disclose an arrangement of a plurality of cell cultures and/or cell agglomerations.

With respect to the method, this task has been solved with the subject matter of claim 1 and an arrangement according to the invention is subject of claim 14. Advantageous embodiments are the subject of the dependent claims and are explained below.

In a method for producing and/or arranging cell cultures and/or cell agglomerations according to the invention, a printing medium containing living cells is provided and the printing medium is printed through a printing screen and/or a printing stencil.

Cell cultures and/or cell agglomerations generated and/or arranged by a method according to the invention are particularly suitable for conducting medical and/or pharmacological investigations.

Printing the print medium through a printing screen and/or a printing stencil can advantageously ensure a high level of productivity. In particular, the design of the printing screen and/or printing stencil allows several defined print areas or print sections to be printed in just one printing step. The entire printing process can thus be completed in only a short period of time. The remaining examination period of the respective cell culture and/or cell agglomeration can thus be extended.

At the same time, the printing screen or printing stencil can advantageously reduce the shear stresses on the cells in the printing medium. In particular, openings in the printing screen and/or printing stencil can be of sufficient size to reduce shear stresses. The risk of damage to the cells in the printing medium as a result of the printing or application process can thus be reduced. The suitability of the cell culture and/or cell agglomeration for medical and/or pharmacological examinations is thereby improved.

Within the meaning of the present invention, a printing screen may be provided with a fabric or formed by a fabric and/or by a frame for the fabric. Such a fabric may be configured for pressing a printing paste therethrough. The fabric of a printing screen may be arranged or clamped in a frame. Further, a fabric may be formed by metal and/or plastic threads. Such metal and/or plastic threads can be arranged twisted or run individually.

To achieve the respective desired printing shape, a printing screen can have permeable and impermeable areas. Permeable regions may be permeable to a printing medium and impermeable regions may be impermeable to a printing medium. Permeable regions of a printing screen, within the meaning of the present invention, may form a passage and/or a cutout in which a fabric permeable to a printing medium is disposed. Impermeable regions may also be provided with a fabric, which, however, is designed to be impermeable to a printing medium or is covered to achieve impermeability.

Fine structures can be produced with a printing screen, in particular with a thickness between 15 μm and 500 μm. Printing by means of a printing screen can be carried out with relatively high accuracy, in particular with high accuracy in the boundary areas of the printed product.

Within the meaning of the present invention, a printing stencil may be provided with openings and/or cutouts, in particular with through-openings and/or cutouts made in a plate. A plate with openings and/or cutouts may form a printing stencil within the meaning of the present invention. Openings and/or cutouts may accordingly form a passage for printing paste. Printing paste can be forced through such openings and/or cutouts as part of a printing process. In particular, a printing stencil can be formed free of fabric. In particular, the openings and/or cutouts of a printing stencil can be free of fabric.

At the same time, it is possible for a form element to be arranged within an opening and/or a cutout of a printing stencil in order to define the shape of the opening and/or the cutout in accordance with the respectively desired printing shape. Such form elements can be held in the desired position within the respective opening and/or cutout by means of individual holding threads or the like.

Printing stencils are particularly suitable for printing layers with a thickness from 100 μm, especially from 300 μm, and particularly preferably from 500 μm. Printing by means of a printing stencil can be carried out at a relatively high speed, since greater application thicknesses can be achieved.

According to an advantageous embodiment, a carrier structure can be provided for a plurality of cell cultures and/or cell agglomerations separated from each other. Furthermore, the printing medium can be printed through a printing screen and/or through a printing stencil onto a carrier structure. In particular, the carrier structure may form a printing substrate onto which the printing medium is printed after passing through the printing screen and/or through the printing stencil. Such a carrier structure can simplify the handling of the cell cultures and/or cell agglomerations, in particular after printing has been completed.

It is also possible for a carrier structure to be printed together with and/or alternately with the printing medium containing living cells and be thereby provided. This can ensure a high degree of manufacturing flexibility. The positioning and alignment of a finished carrier structure below a printing screen and/or below a printing stencil can thus be omitted. The preparation effort for printing can thus be reduced.

Likewise, it is possible for a carrier structure to be printed prior to printing the print medium containing living cells and to be provided in this way. The fully printed carrier structure can then be used as a printing substrate.

According to a further advantageous embodiment, single-layer or multilayer cell agglomerations and/or single-layer or multilayer cell cultures can be generated by printing the printing medium. The printing of a single-layer cell agglomeration and/or cell culture can be realized with only little effort and thus at low cost. Multi-layered cell agglomerations and/or cell cultures enable particularly meaningful examinations, since the conditions of living organisms can be particularly well reproduced by multi-layered formation.

In a further preferred manner, a multilayered tissue can be easily and quickly produced by repeated printing of a printing medium containing living cells and/or by varying the cells or the printing medium containing cells in a layer build-up direction (z-direction). Such a multilayer tissue can be, for example, skin or other types of cell tissue.

In a further preferred manner, the printing medium can be fed through the printing screen and/or through the printing stencil and/or onto a carrier structure by at least one blading operation and/or at least one printing blade movement, in particular by a plurality of blading operations and/or a plurality of printing blade movements. Blading operations or printing blade movements permit rapid and uniform printing of the printing medium through the printing screen or through the printing stencil. In particular, this allows specifically reproducible results to be achieved.

In a particularly preferred manner, the print medium can be printed by at least one screen printing process, in particular by a plurality of screen printing processes. In particular, the printing of the print medium can be carried out by a screen printing process and/or a stencil printing process, especially preferably by a 2D screen printing process or a 3D screen printing process. This can ensure a particularly high level of productivity and accuracy. In the screen printing process, relatively large volumes of a print medium can be printed with only small efforts of time and costs. The arrangement and/or generation of cell cultures and cell agglomerations can thus be carried out quickly and cost-effectively. At the same time, the use of a screen printing process, in particular a 2D screen printing process or 3D screen printing process, ensures gentile processing of the printing medium containing living cells. The respective cells can be processed and/or printed in the screen printing process with only little risk of damage due to shear stresses. In particular, a 3D screen printing system can be used for printing the print medium.

In a further preferred manner, substructures of the cell cultures and/or cell agglomerations or tissue produced therefrom are printed with an accuracy of up to 100 cells, preferably up to 50 cells, in particular 20 cells, further preferably 10 cells, in particular printed with an accuracy of up to a single cell. Such printing or manufacturing accuracies can be achieved both in the layer build-up direction (z-direction) and/or in directions transverse to the layer build-up direction (x/y-directions). In particular, such accuracies can be achieved by means of 3D screen printing.

The screen printing process or the plurality of screen printing processes can advantageously be carried out onto, in particular directly onto, a carrier structure. Screen printing onto a carrier structure, for example onto a cell carrier, enables a high degree of reproducibility and ease of handling when removing the printed cells after completion of the printing process.

It can also be advantageous if the printing of the printing medium containing living cells is carried out together and/or alternately with the printing of the carrier structure in a 2D screen printing process or 3D screen printing process. This enables a flexible design of the carrier structure as well as of the entire printing process. The desired shape of the carrier structure can thus be generated as required.

In a still further preferred embodiment, a three-dimensional cell culture and/or cell agglomeration can be generated by printing a carrier structure with the printing medium. Three-dimensional cell cultures and/or cell agglomerations can be generated in particular by multiple printing or by 3D screen printing.

Furthermore, it is possible that after printing a carrier structure with the printing medium, the respective cell culture and/or cell agglomeration develops into a cell culture and/or cell agglomeration, in particular a three-dimensional cell culture and/or cell agglomeration. Accordingly, a development time may be required between the completion of printing and the development of a cell culture and/or cell agglomeration. The properties and/or characteristics of the cell cultures or cell agglomerations can be influenced by the duration of such a development time.

According to a further preferred embodiment, structural matrixes and/or scaffolds may be provided in the printing sections, i.e. the sections generated by printing the printing medium containing living cells. Likewise, structural matrixes and/or scaffolds may be printed together with and/or contained within the printing medium. Matrixes and/or scaffolds are particularly advantageously suited for the generation of three-dimensional structures. Cell cultures and/or cell agglomerations can be formed three-dimensionally in this way with only little effort and at the same time reproducibly.

When printing the print medium containing living cells, cavities, recesses, linear or channel-shaped structures can be provided in a particularly advantageous manner. Such shapes allow improved functionalization of the cell cultures, cell agglomerations and/or tissues produced with the printing medium. By means of such cavities, recesses and/or structures, for example, scaffolds and/or blood vessels can be formed.

In a particularly preferred manner, a combination of materials can be printed, especially in order to produce scaffolds and/or matrices with cells in a single printing process with only little effort.

According to a further preferred embodiment, the respective cell culture and/or cell agglomeration may develop into tissue after printing. Tissue development may be particularly advantageous for use in medical or pharmacological examinations. Similarly, such developed tissue may be used for medical treatment. Finally, such developed tissue may serve as food. In particular, the respective cell culture and/or cell agglomeration may develop into synthetic meat after printing, which may be suitable for consumption.

According to a further advantageous embodiment, the carrier structure can be designed for arranging and/or receiving a plurality of cell cultures and/or cell agglomeration separated from one another. In particular, the carrier structure can have predefined arrangement and/or receiving sections for cell cultures and/or cell agglomeration separated from one another. This can ensure a secure and defined arrangement of the printed cells or cell cultures and/or cell agglomeration formed by the cells on the carrier structure.

In a preferred manner, the arranging and/or receiving sections may be provided in a plurality of lines and rows on the carrier structure. In particular, the number of lines may be different from the number of rows. The outer shape of the carrier structure may be adapted to the number of rows and lines. Further preferably, the carrier structure may have recesses for receiving the printing medium and/or the arrangement and/or receiving sections may be formed as recesses.

In a particularly preferred manner, the carrier structure can be designed as an object carrier and/or cell culture plate and/or cell carrier. The carrier structure is thus particularly advantageously suited for use and/or handling in laboratories or for standardized transport containers, transport devices and/or storage devices.

In a further preferred manner, the printing screen and/or the printing stencil can have a plurality of cutouts for the passage of the printing medium, in particular cutouts formed separately from one another. This allows a defined passage of the printing medium through the printing screen and/or the printing stencil. In particular, the print medium can be printed and/or applied to a carrier structure in a defined manner at different positions and/or positions spaced-apart from each other.

In a further preferred manner, the cutouts of the printing screen and/or the printing stencil can correspond to predefined arrangement and/or receiving sections of the carrier structure. In this way, the printing medium can be printed specifically in and/or on the arrangement and/or receiving sections of the carrier structure. In particular, such an embodiment can prevent accidental or undesired printing onto the carrier structure outside the arrangement and/or receiving sections. At the same time, a high printing volume per printing process can be realized.

According to a further preferred embodiment, at least one cutout and/or passage of the printing screen and/or the printing stencil for the passage of the printing medium may have a size and/or diameter of at least 1 mm, in particular of at least 2, preferably of at least 2 mm, preferably of at least 3 mm, more preferably of 4 mm, more preferably of at least 5 mm.

According to a still further preferred embodiment, at least one cutout and/or passage of the printing screen and/or of the printing stencil for the passage of the printing medium may have a size and/or a diameter of up to 100 mm, in particular of up to 50 mm, preferably of up to 40 mm, preferably of up to 30 mm, further preferably of up to 25 mm, further preferably of up to 20 mm, still further preferably of up to 15 mm, further preferably of up to 10 mm, still further preferably of up to 9 mm, further preferably of up to 8 mm, still further preferably of up to 5 mm.

According to a further preferred embodiment, at least one cutout and/or passage of the printing screen and/or the printing stencil for the passage of the printing medium may have a size and/or diameter of from 1 mm to 100 mm, in particular from 2 mm to 50 mm, preferably from 2 mm to 40 mm, more preferably from 2 mm to 30 mm, further preferably from 3 mm to 30 mm, further preferably from 3 mm to 25 mm, further preferably from 3 mm to 20 mm, still further preferably from 4 mm to 20 mm, further preferably from 4 mm to 15 mm, further preferably from 4 mm to 10 mm, still further preferably from 4 mm to 9 mm, further preferably from 4 mm to 8 mm, still further preferably from 5 mm to 10 mm, further preferably from 5 mm to 8 mm. By means of such a dimensioned cutout and/or passage in the printing screen and/or in the printing stencil, on the one hand only a low shear stress of the living cells in the printing medium and at the same time also a relatively large printing volume per printing process can be ensured.

According to a preferred embodiment, a plurality of arrangement and/or receiving sections of the carrier structure can be printed onto and/or filled with the print medium simultaneously and/or in one printing process and/or by one blading operation. This can be accomplished with only little effort. In particular, all arrangement and/or receiving sections of the carrier structure can be printed onto with the printing medium in one printing process. The sequential filling of individual receiving sections in many individual steps can thus be avoided.

It is also possible for different arrangement and/or receiving sections of the carrier structure to be printed onto and/or filled in chronological sequence according to their distance from one another and according to the respective blading speed. The individual arrangement and/or receiving sections of the carrier structure can thus be filled in accordance with a doctor blade movement. This means that the arrangement and/or receiving sections do not have to be filled at the same time, which increases process flexibility.

According to a further preferred embodiment of the process according to the invention, the print medium can be printed at individual positions on a carrier structure. By printing the print medium onto a carrier structure, print sections that are independent of one another and/or fluid-technically separated can be generated in a particularly advantageous manner. As a result, several cell cultures and/or cell agglomerations independent of each other can be generated, which are available for a relatively large number of medical and/or pharmacological examinations.

It may be further advantageous to superimpose a sterile gas medium on the printing medium during printing, before printing and/or after printing, in particular sterile air. Sterility requirements for handling and/or processing living cells can be safely followed in this way. Unwanted contamination of the cell cultures and/or cell agglomerations can thus be avoided.

The printing medium can in particular be a nutrient medium and/or a medium containing nutrients, in particular a nutrient liquid. Such a printing medium or nutrient medium can be designed in particular as a so-called bio ink.

According to a still further preferred embodiment, the printing medium can be in the form of a printing paste and/or low-viscosity or medium-viscosity nutrient liquid and/or liquid suspension. In purely gel-like media, cells and/or nutrients can only diffuse to a limited extent, which can lead to the availability of nutrients being limited and thus also to the lifespan of the cells being limited. By forming the printing medium as a relatively low-viscosity printing paste and/or low-viscosity or medium-viscosity nutrient liquid and/or liquid suspension, a high degree of nutrient supply to the living cells can be ensured within the printing medium.

It is also possible for the printing medium to be in the form of a gel and/or a highly viscous nutrient medium, which can simplify processing or printing.

Furthermore, the printing medium can be designed as a sol-gel matrix and/or have a variable viscosity and/or the viscosity of the printing medium can be changed by a drying step and/or a tempering step, in particular a temperature reduction or temperature increase. Depending on the processing step performed, the viscosity can thus be changed. In this way, a high level of nutrient supply can be ensured over relatively long periods of time and, at the same time, the viscosity can be changed for the purpose of processing or further processing steps.

The printing medium can also be designed to be structurally viscous and/or shear-thinning. In the case of relatively high shear stresses on the printing medium, this can result in a temporarily lower viscosity and thus reduce the shear stress. This further reduces the risk of cell damage.

In a further preferred manner, a drying and/or tempering step of the printed printing medium can take place between successive printing steps. It is also possible that a sol-gel transition is generated after the printing of the print medium, in particular by a drying and/or tempering step, and/or that a further printing process is carried out after a generated sol-gel transition. The properties of the printing medium or of the cell culture and/or cell agglomeration produced as a result can thus be specifically influenced as a function of the respective process stage.

In a further preferred way, the printing medium can be liquid or have a low viscosity during printing, so that simple printing is possible. Following the printing or the printing of a printing layer, the respective printing layer can be solidified or the viscosity of the printed medium can be increased, for example by tempering and/or by generating a sol-gel transition. The respective printed object or the cell culture and/or cell agglomeration generated by printing can thus be built up in a suitable manner in a layer build-up direction (z-direction).

As soon as the respective print object is built up in height (z-direction), it can be inserted into a suitable mold and/or stencil or the mold and/or stencil can be placed on the print object to surround it. The printed object can thus be limited in the x/y direction. The printed medium or the cell culture and/or cell agglomeration produced by printing can be returned to a more fluid state or to a state with lower viscosity, respectively, without melting. This allows nutrients to circulate freely again within the cell culture and/or cell agglomeration produced by printing. Tissue can develop in an improved manner.

In a further preferred embodiment, the printing medium may contain cells capable of division and/or cells inducible to division. Further, the living cells of the printing medium may be human, animal and/or plant cells. In particular, the printing medium may contain all types of human, animal and/or plant cells. Particularly preferably, the living cells may be all cells of the human or animal body or of plants that are capable of division or can be induced to divide. This results in a particular suitability for medical and pharmacological examinations.

In a further preferred manner, the printing medium may comprise living cells from the group of primary cells, in particular all types of human, animal and/or plant primary cells.

Similarly, the printing medium may comprise living cells from the group of cell lines, in particular all types of human, animal and/or plant established cell lines.

Primary cells may be, for example, organ cells, in particular organ cells of all organs, skin cells, fibroblasts, chondroblasts, osteoblasts, muscle cells, cardiac muscle cells, nerve cells, liver cells, islet cells, vascular cells, glandular tissue cells, tumor cells, in particular tumor cells of all tumor tissues, stem cells, in particular hematopoietic, mesenchymal and/or neuronal stem cells and/or induced pluripotent stem cells from tissues, such as fat tissue, skin and/or umbilical cord, and/or totipotent stem cells, in particular ovules and/or embryonic cells, and/or pluripotent, multipotent, oligopotent and/or unipotent stem cells, blood cells and/or immune cells.

Cell lines may be, for example, immortalized cells of the above-mentioned cell types and/or cells of the above-mentioned tissue types.

It may be further advantageous if the printing medium contains different cells and/or cell types, in particular different cells and/or cell types as mentioned above. This can result in a further improved suitability of the cell cultures and/or cell agglomerations produced for medical and/or pharmacological examinations.

Further preferably, a tissue produced from the printing medium may contain different cells and/or cell types, in particular different cells and/or cell types as mentioned above. This can also result in a further improved suitability of the generated tissue for medical and/or pharmacological examinations.

It may be further advantageous if the printing medium contains at least one growth factor and/or at least one protein and/or extracellular matrix protein, in particular a growth factor and/or protein from the group of cytokines, in particular as growth regulators, interferons and/or interleukins, membrane components, laminins, collagens, in particular collagen type 4, proteoglycans, entactins, nidogens, cell adherence factors, in particular fibronectin and/or vitronectin, growth factors, in particular of the EGF family, the TGF family, PDGF, VEGF, somatomedins, in particular IGF, NGF, PTGF, and/or protective factors, in particular tissue-specific plasminogen activators, serum albumin and/or CMC. In a particularly preferred manner, the printing medium may comprise at least one or more of these substances from the aforementioned groups. This can result in further improved suitability of the cell cultures, cell agglomerations and/or tissues produced by means of the printing medium for carrying out medical and/or pharmacological examinations.

In a further preferred manner, the printing medium may have cells with a size of 5-50 μm, 5-40 μm, 10-50 μm, 10-40 μm, 10-30 μm, 20-40 μm, 25-40 μm, 25-30 μm in diameter or in cross-sectional length. Furthermore, a cell culture can have a number of 5 to 1000 cells, in particular 10 to 1000 cells, 20 to 500 cells, 50 to 500 cells, 100 to 500 cells, 200 to 500 cells or 300 to 500 cells. This allows particularly meaningful examinations to be carried out by means of the cell cultures and/or cell agglomerations produced by the printing medium.

Another independent aspect of the present invention relates to a method for generating and/or arranging cell cultures and/or cell agglomerations, wherein a carrier structure for a plurality of cell cultures separated from each other is provided and a printing medium containing living cells is printed onto the carrier structure by screen printing.

A still further independent aspect of the present invention relates to an arrangement of a plurality of cell cultures and/or cell agglomerations printed on a carrier structure by a method described above.

A still further independent aspect of the present invention relates to an arrangement of a plurality of tissues and/or tissue sections produced at least by printing a printing medium according to a method described above onto a carrier structure and subsequent and/or preceding cell cultivation.

According to a particularly preferred embodiment, the cell cultures and/or cell agglomerations and/or tissues may be the same with respect to the metabolic state of the cells, the cell age and/or the number of cells and/or the dimension of the number of cells. Likewise, it is possible for the cell cultures and/or cell agglomerations and/or tissues to have deviations with respect to the metabolic state of the cells, the cell age and/or cell number that are less than 20%, in particular less than 10%, less than 5%, less than 3%, less than 2% or less than 1%. By such an arrangement, the cell cultures and/or cell agglomerations and/or tissues can serve for a variety of examinations in which the examination parameters can be varied between the individual cell cultures and/or cell agglomerations and/or tissues in a suitable manner. Such examination series can be particularly informative.

For the purpose of this document, tissue may be understood to mean, in particular, cellular tissue.

### BRIEF DESCRIPTION OF THE DRAWINGS

Further embodiments of the present invention result from combinations of the features disclosed in the claims, the description and the figures. The present invention is explained below with reference to embodiments and associated drawings.

It is shown schematically in each case:

FIG. 1 a carrier structure for cell cultures and/or cell agglomerations and a printing screen or printing stencil arranged above it in perspective view,

FIG. 2 an arrangement of a plurality of cell cultures and/or cell agglomerations on a carrier structure according to FIG. 1 in perspective view.

### DETAILED DESCRIPTION

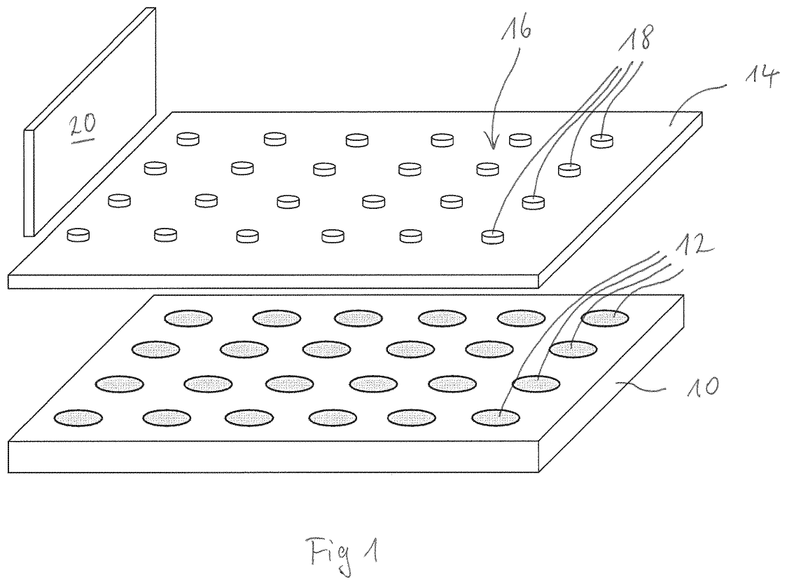

FIG. 1 shows a carrier structure 10 for cell cultures and/or cell agglomerations in perspective view. The carrier structure 10 can be, in particular, a cell culture plate, an object carrier or a so-called array for cell cultures and/or cell agglomerations. The carrier structure 10 can have a plurality of receiving sections 12.

The receiving sections 12 may be configured to receive cell cultures and/or cell agglomerations not shown in more detail herein. In particular, a printing medium containing living cells, not shown in greater detail herein, may be fed into the receiving sections 12 by printing, as will be explained in greater detail below. Once such a printing medium is disposed in the receiving sections 12, it may develop into cell cultures and/or cell agglomerations. Similarly, a printing medium already consisting of cell cultures and/or cell agglomerations and/or containing cell cultures and/or cell agglomerations may be arranged in the receiving sections 12 by printing.

According to the exemplary embodiment in FIG. 1, the receiving sections 12 can be formed by recesses, in particular by bowl-shaped and/or hemispherical and/or cylindrical and/or rectangular recesses. The carrier structure 10 shown by way of example in FIG. 1 has a total of 24 receiving sections 12. Exemplarily, the receiving sections 12 may be formed in a total of 4 lines and 6 rows in the carrier structure 10. Of course, carrier structures 10 with a different number of receiving sections 12 or with other arrangements of receiving sections 12 may also be suitable.

FIG. 1 further shows a printing stencil 14 in schematic perspective view. The printing stencil 14 may be formed by a plate 16 having a plurality of cutouts 18 for the passage of a printing medium. In particular, the cutouts 18 are formed separately from one another.

Instead of the printing stencil 14, a printing screen not shown in more detail here can also be provided, which is equipped with a fabric. Within such a fabric, areas can be provided which are permeable to printing paste and are bounded by impermeable areas. Permeable areas are also referred to herein as cutouts, although these may have a fabric.

The cutouts 18 of the printing stencil 14 or of a printing screen not shown here can, for example, have a diameter of at least 1 mm, in particular of at least 2 mm, preferably of at least 3 mm, more preferably of at least 4 mm, more preferably of at least 5 mm.

In particular, the cutouts 18 of the printing stencil 14 or of a printing screen not shown here may have a diameter of from 1 mm to 100 mm, in particular from 2 mm to 50 mm, preferably from 2 mm to 40 mm, preferably from 2 mm to 30 mm, more preferably from 3 mm to 30 mm, more preferably from 3 mm to 25 mm, further preferably from 3 mm to 20 mm, still further preferably from 4 mm to 20 mm, further preferably from 4 mm to 15 mm, further preferably from 4 mm to 10 mm, still further preferably from 4 mm to 9 mm, further preferably from 4 mm to 8 mm, still further preferably from 5 mm to 10 mm, further preferably from 5 mm to 8 mm. The cutouts 18 of the printing stencil 14 may have different diameters or identical diameters.

Furthermore, the cutouts 18 of the printing stencil 14 or of a printing screen not shown here can correspond in a particularly preferred manner with the predefined receiving sections 12 of the carrier structure 10.

In accordance with the present invention, a printing medium containing living cells is printed through a printing screen and/or through the printing stencil 14.

For printing the printing medium, the printing screen and/or the printing stencil 14 can be arranged above the carrier structure 10, as shown in FIG. 1. The printing blade 20 further shown schematically in FIG. 1 can be used for printing a printing medium through the printing screen and/or through the printing stencil 14. Thereby, the printing medium can be printed through the printing screen and/or the printing stencil 14 by at least one blading operation and/or at least one printing blade movement, in particular by a plurality of blading processes of the printing blade 20 and/or a plurality of printing blade movements of the printing blade 20. In this way, the printing medium can be fed onto the carrier structure 10 in a particularly reliable manner.

Printing of the print medium by means of the printing screen and/or the printing stencil 14 and/or the printing blade 20 can be carried out in particular using a screen printing process. The screen printing process can in particular be a 2D screen printing process or 3D screen printing process.

It is further possible that the carrier structure 10 is printed together and/or alternately with the printing medium containing living cells. In particular, printing of the living cell-containing print medium may be performed together and/or alternately with printing of the carrier structure 10 using a 3D screen printing process. Further, the carrier structure 10 may be printed prior to printing the live cell-containing print medium, particularly using a 3D screen printing process. Likewise, the carrier structure 10 may be otherwise manufactured and provided prior to printing the living-cell-containing print medium.

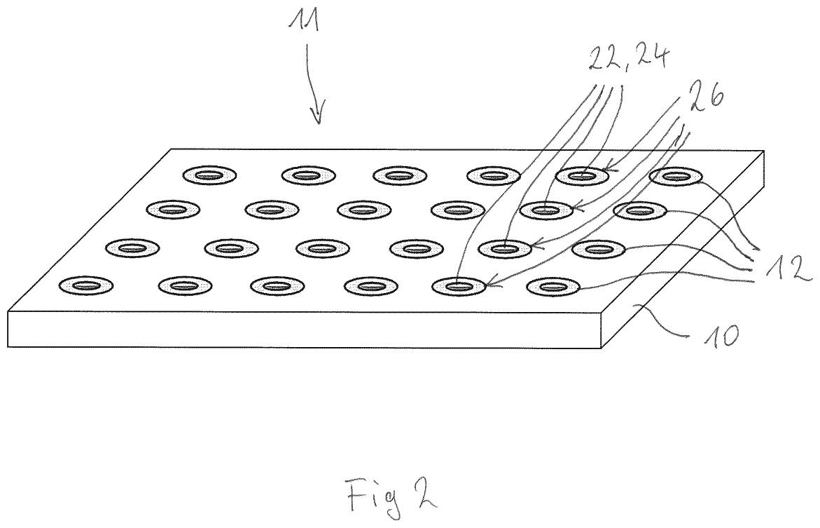

In FIG. 2, an arrangement 11 of a plurality of cell cultures 22 and/or cell agglomerations 24 on a carrier structure 10 is shown in perspective view. Each of the cell cultures 22 and/or cell agglomerations 24 is arranged in one of the receiving sections 12. Single or multi-layered cell cultures 22 and/or single or multi-layered cell agglomerations 24 may be produced by printing the printing medium, which is not shown in detail herein. Further, by printing the printing medium on the carrier structure 10, a three-dimensional cell culture 22 and/or cell agglomeration 24 can be generated. After printing the carrier structure 10 with the printing medium, the respective cell culture 22 and/or cell agglomeration 24 may develop into a three-dimensional cell culture 22 and/or cell agglomeration 24.

It is also possible that structural matrices and/or scaffolds are provided in printing sections 26 or receiving sections 12, which are also not shown in more detail here. Such structural matrices and/or scaffolds can be printed together with the printing medium and/or be contained therein.

In a particularly preferred manner, a plurality of receiving sections 12 of the carrier structure 10 can be printed and/or filled with the printing medium simultaneously and/or in one printing process and/or by one blading process. Likewise, different receiving sections 12 of the carrier structure 10 can be printed and/or filled in temporal sequence according to their distance from each other and according to the respective printing blade speed.

By printing the carrier structure 10 with the printing medium, printing sections 26 that are independent of one another and/or fluid-technically separate can be generated. In particular, one printing section 26 can be generated for each receiving section 12. A printing section 26 may form or develop into a cell culture 22 and/or a cell agglomeration 24. Further, the cell cultures 22 and/or the cell agglomerations 24 may develop into tissue after printing.

In the arrangement 11 shown in FIG. 2, the individual cell cultures 22 and/or cell agglomerations 24 may be the same with respect to the metabolic state of the cells, the cell age and/or the number of cells and/or the dimension of the number of cells. Likewise, it is possible that in the arrangement 11 according to FIG. 2, deviations with respect to the metabolic state of the cells, the cell age and/or the number of cells are less than 20%, in particular less than 10%, less than 5%, less than 3%, less than 2% or less than 1%.

The printing medium or the cell cultures 22 and/or cell agglomerations 24 produced therewith may contain cells capable of division and/or cells that can be induced to divide. The living cells of the printing medium and/or the cell cultures 22 and/or cell agglomerations 24 may be human, animal and/or plant cells. In particular, the printing medium and/or the cell cultures 22 and/or cell agglomerations 24 may contain all types of human, animal and/or plant cells. Particularly preferably, the living cells of the printing medium and/or the cell cultures 22 and/or cell agglomerations 24 may be all cells of the human or animal body or of plants that are capable of dividing or can be induced to divide.

Furthermore, the printing medium or a cell culture 22 and/or cell agglomeration 24 generated therefrom may comprise living cells from the group of primary cells, in particular all types of human, animal and/or plant primary cells. Likewise, the printing medium or a cell culture 22 and/or cell agglomeration 24 generated therefrom may comprise living cells from the group of cell lines, in particular all types of human, animal and/or plant established cell lines. Finally, the printing medium and/or a cell culture 22 and/or cell agglomeration 24 generated therefrom may contain different cells and/or cell types.

Furthermore, the printing medium or a cell culture 22 and/or cell agglomeration 24 produced therefrom may contain at least one growth factor and/or at least one protein and/or extracellular matrix protein, in particular a growth factor and/or protein from the group of cytokines, in particular as growth regulators, interferons and/or interleukins, membrane components, laminins, collagens, in particular collagen type 4, proteoglycans, entactins, nidogens, cell adherence factors, in particular fibronectin and/or vitronectin, growth factors, in particular of the EGF family, the TGF family, PDGF, VEGF, somatomedins, in particular IGF, NGF, PTGF, and/or protective factors, in particular tissue-specific plasminogen activators, serum albumin and/or CMC. Likewise, the printing medium and/or a cell culture 22 and/or cell agglomeration 24 produced therefrom may comprise at least one or more of these substances from the aforementioned groups.

The cell cultures 22 and/or cell agglomerations 24 produced according to the methods described above are particularly preferably suitable for conducting medical and/or pharmacological examinations.

1. A method for producing and/or arranging cell cultures (22) and/or cell ag-glomerations (24) for carrying out medical and/or pharmacological examinations, in which a printing medium containing living cells is provided and in which the printing medium is printed through a printing screen and/or a printing stencil (14).

2. The method according to claim 1, wherein:
the printing medium is printed through a printing screen and/or a printing stencil (14) onto a carrier structure (10), and/or
a carrier structure (10) is provided for a plurality of cell cultures (22) and/or cell agglomerations (24) separated from each other, and/or
a carrier structure (10) is printed together and/or alternately with the printing medium containing living cells, and/or
a carrier structure (10) is printed before printing the printing medium containing living cells.

3. The method according to claim 1,
wherein the printing medium is conveyed through the printing screen and/or the printing stencil (14) and/or onto a carrier structure (10) by at least one blading operation and/or at least one printing blade movement.

4. The method according to claim 1, wherein:
the printing medium is printed by at least one screen printing process in particular on a carrier structure (10), and/or
the printing of the printing medium is carried out in a screen printing process, and/or
the printing of the printing medium containing living cells is carried out together and/or alternately with the printing of the carrier structure (10) in a screen printing process, and/or
single-layer or multilayer cell cultures (22) and/or single-layer or multilayer cell agglomerations (24) are produced by the printing of the printing medium.

5. The method according to claim 1, wherein:
by printing the printing medium onto a carrier structure (10) a three-dimensional cell culture (22) and/or cell agglomeration (24) is generated, and/or
after printing the printing medium onto a carrier structure (10), the respective cell culture (22) and/or cell agglomeration (24) develops into a three-dimensional cell culture (22) and/or cell agglomeration (24), and/or
structural matrices and/or scaffolds are provided in printing sections (26), and/or
structural matrices and/or scaffolds are printed together with the printing medium and/or are contained therein, and/or
the cell cultures (22) and/or the cell agglomerations (24) develop into tissue after printing.

6. The method according to claim 2, wherein:
the carrier structure (10) is designed for arranging and/or receiving a plurality of cell cultures (22) and/or cell agglomeration (24) separated from one another, and/or in
the carrier structure (10) has predefined arrangement and/or receiving sections (12) for cell cultures (22) and/or cell agglomerations (24) separated from one another, and/or that
the arrangement and/or receiving sections (12) are provided in a plurality of lines and rows on the carrier structure (10), and/or
the carrier structure (10) is designed as an object carrier and/or cell culture plate and/or comprises depressions for receiving the printing medium.

7. The method according to claim 1, wherein the printing screen and/or the printing stencil (14) has a plurality of cutouts (18) for the passage of the printing medium, the plurality of cutouts (18) are formed separately from one another.

8. The method according to claim 1, wherein at least one cutout (18) of the printing screen and/or the printing stencil (14) for the passage of the printing medium has a size and/or a diameter from 1 mm to 100 mm.

9. The method according to claim 2, wherein:
a plurality of arrangement and/or receiving sections (12) of the carrier structure (10) are printed and/or filled with the printing medium simultaneously and/or in a printing process and/or by a blading operation, and/or
the printing medium is printed at individual positions on a carrier structure (10), and/or
by printing the printing medium onto a carrier structure (10) printing sections (26) are produced, which are independent and/or fluid-technically separated from one another.

10. The method according to claim 1, wherein during printing, before printing and/or after printing, superimposition of a sterile gas medium on the printing medium is conducted.

11. The method according to claim 1, wherein:
the printing medium is formed as printing paste and/or low-viscosity or medium-viscosity nutrient liquid and/or liquid suspension and/or sol-gel matrix, and/or
the printing medium has a variable viscosity, and/or in
the viscosity of the printing medium can be changed by a drying step and/or a tempering step, and/or
the printing medium is structurally viscous.

12. The method according to claim 1, wherein:
a drying and/or tempering step of the printed printing medium is carried out between successive printing steps, and/or
a sol-gel transition is generated after the printing of the printing medium by a drying and/or tempering step, and/or
a further printing process is carried out after a generated sol-gel transition.

13. The method according to claim 1, wherein a carrier structure (10) for a plurality of cell cultures (22) and/or cell agglomerations (24) being separated from each other is provided and a printing medium containing living cells is printed onto the carrier structure (10) by means of screen printing.

14. An arrangement (11) of a plurality of cell cultures (22) and/or cell agglomerations (24) printed on a carrier structure (10) by the method according to claim 1.

15. An arrangement according to claim 14, wherein the cell cultures (22) and/or cell agglomerations (24) are equal with respect to the metabolic state of the cells, the cell ageU, the cell number, the dimension of the cell numberl, and/or deviations with respect to the metabolic state of the cells, and the cell age and/or the cell number are less than 20.

16. The method according to claim 2, wherein the printing screen and/or the printing stencil (14) has a plurality of cutouts (18) for the passage of the printing medium and the cutouts (18) of the printing screen and/or the printing stencil (14) correspond to a predefined arrangement and/or receiving sections (12) of the carrier structure (10).
